(19) United States
(12) Patent Application Publication
(10) Pub. No.: US 2023/0029928 A1
(43) Pub. Date: Feb. 2, 2023

(54) INTERMITTENT CATHETERS

(71) Applicant: ConvaTec Limited, Deeside (GB)

(72) Inventors: Lukas Kandrac, Michalovce (SK); David Pollard, Deeside, Flintshire (GB); Rachel Zimet Pytel, Las Vegas, NV (US)

(21) Appl. No.: 17/871,406

(22) Filed: Jul. 22, 2022

Related U.S. Application Data
(63) Continuation of application No. PCT/GB2022/051925, filed on Jul. 22, 2022.
(60) Provisional application No. 63/203,598, filed on Jul. 27, 2021.

(51) Int. Cl. A61M 25/00; A61L 29/14; A61L 29/04; B29C 48/00; B29C 48/09; B29C 48/21; B29C 33/60

(52) U.S. Cl. CPC A61M 25/0045; A61L 29/14; A61L 29/041; A61M 25/0009; B29C 48/022; B29C 48/09; B29C 48/21; B29C 33/60; A61M 2025/0046; A61L 2420/02; A61L 2400/10; B29L 2031/7542

(57) ABSTRACT

The invention provides an intermittent catheter comprising a hollow polymeric tubular body comprising a base polymer and a layer comprising a lubricious additive on or comprising a surface of the body, wherein the lubricious additive comprises an amphiphilic molecule.

### CROSS REFERENCE TO RELATED DISCLOSURES

The present disclosure is a continuation of International Application No. PCT/GB2022/051925 filed on July 22, 2022 and claims the benefit of U.S. Provisional Application No. 63/203,598 filed on July 27, 2021, the disclosure of which are incorporated herein in entirety.

### TECHNICAL FIELD OF THE INVENTION

The present invention relates to intermittent catheters comprising a base polymer and a layer comprising a lubricious additive comprising an amphiphilic molecule, and to methods of manufacturing said intermittent catheters.

### BACKGROUND TO THE INVENTION

Intermittent urinary catheterisation is a process involving insertion of a urinary catheter through an individual's urethra and into their bladder, where it is retained to empty the bladder of urine for only the time period that is required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Intermittent catheterisation is often used by patients suffering from abnormalities of the urinary system, resulting in urinary incontinence and/or a lack of control in permitting voluntary urination. Such individuals would typically make use of intermittent catheters several times a day.

Intermittent catheters are useful devices, providing users with independence and freedom to self-catheterise as and when required, without having to rely on trained personnel to be present. This, however, increases the need for intermittent catheters to be user friendly: in particular, both easy to insert and remove with minimum discomfort caused, and safe to use with features for minimising risk of infection. Users often report experiencing pain and discomfort upon insertion and/or removal of intermittent catheters. Users have, for instance, reported experiencing bladder spasms, burning sensations, and bleeding. Urinary tract infections (UTI) are also common in individuals who practice intermittent catheterisation.

Surface coatings and additives for catheters have been used to help in alleviating these issues. US patents U.S. Pat. Nos. 10,058,638 B2 and 9,186,438 B2 describe the use of a catheter containing a polymer mixture of a thermoplastic or thermo-curing polymer base material and an amphiphilic block copolymer lubricious additive. The amphiphilic block copolymer contains both a hydrophobic and hydrophilic portion. The hydrophilic portion diffuses to the surface of the catheter due to incompatibility with the hydrophobic base material and provides for a lubricious surface coating. Interactions between the hydrophobic portion of the amphiphilic molecule and the base material assist in reducing migration of the amphiphilic molecule from the catheter.

Whilst this method has its advantages, the diffusion of the additives to the surface of the catheter can often be non-ideal, necessitating the use of a large amount of lubricious additive to confer the required lubricity to the surface of the catheter. This can result in significant additive wastage and increased costs of catheter production.

Furthermore, addition of large quantities of additive to the bulk polymer mixture of a catheter can lead to alteration and/or degradation of the mechanical properties of the catheter body, which is naturally undesired.

There is a particular need for lubricious intermittent catheters which can be made both efficiently and well, making them easier and safer to use by non-medically trained individuals.

It is an aim of embodiments of the present invention to address one or more of the above problems by providing an intermittent catheter, suitable for self-catheterisation use, which provides one or more of the following advantages:
A lubricious, non-stick surface making the intermittent catheter easier to insert and remove.
Reduced amount of lubricious additive needed to generate a lubricious intermittent catheter surface.
Reduced cost of production of a lubricious intermittent catheter.
Reduced undesired alteration and/or degradation of mechanical properties of an intermittent catheter.

It is also an aim of embodiments of the invention to overcome or mitigate at least one problem of the prior art, whether expressly described herein or not.

### SUMMARY OF THE INVENTION

According to first aspect of the invention, there is provided an intermittent catheter comprising a hollow polymeric tubular body comprising a base polymer and a layer comprising a lubricious additive on or comprising a surface of the body, wherein the lubricious additive comprises an amphiphilic molecule.

The amphiphilic lubricious additive comprises a hydrophobic portion and a hydrophilic portion. In cases where the base polymer is hydrophobic or generally hydrophobic, such as a polyolefin, the hydrophobic portion of the additive will interact with the hydrophobic base polymer and the hydrophilic portion will seek away from the base polymer and towards the external environment.

The layer comprising a lubricious additive may be on the surface of the body. In some embodiments, the layer comprises a layer comprising an additive that is substantially separate from the body and the layer may be bonded to the body. The layer may be bonded to the body via covalent bonds, ionic bonds, hydrogen bonds, or Van der Waals forces. The lubricious additive may be bonded to the body via one or more surface linker groups which may be present on the additive, the body of the intermittent catheter or both.

In some embodiments, the layer comprising a lubricious additive may comprise the surface of the body. In such embodiments the layer comprising a lubricious additive may form the surface of the body. The layer may comprise a co-extruded layer which is melded with or is physically entangled with the body, and this may form an integral layer. The layer of lubricious additive may be integrally formed with the body.

In some embodiments, polymer diffusion occurs between the layer comprising a lubricious additive and the catheter body. The layer and the body may be held together by polymer chains extending across the interface between the layer and body. In some embodiments, the additive infiltrates the catheter body.

In preferred embodiments, the surface comprises an outer surface of the body. The outer surface may comprise one or more surfaces selected from the group comprising the external surface of the tubular body, the inner lumen and eyelets. The outer surface is preferably the external surface of the tubular body. The outer surface may comprise the external surface of the body and the inner lumen. The outer surface may comprise the external surface of the body and the eyelets. In some embodiments, the outer surface may comprise the external surface of the body, the inner lumen, and the eyelets.

When the layer comprising a lubricious additive comprises or is on an outer surface of the body, it enables wetting of the surface simply by applying water or a wetting agent or by wiping with a wet wipe, to create a lubricious coating, making the intermittent catheter easier and less painful to use, especially for individuals practicing self-catheterisation.

Having a layer comprising a lubricious additive on or comprising the outer surface of the body reduces the amount of additive needed to generate a lubricious intermittent catheter surface, as it removes the need to rely on diffusion of the additive from the bulk polymer mixture of the catheter and towards the outer surface to generate the lubricity. This also allows for reduced amounts of additive to be added to the base polymer mixture of the intermittent catheter, which assists in reducing undesired alteration and/or degradation of the mechanical properties of the intermittent catheter body brought about by the additive, during manufacture and/or use.

In some embodiments, the layer comprising an additive comprises or is on an inner surface of the body, an outer surface of the body, or both. The inner surface of the body may comprise a lumen of the intermittent catheter. In preferred embodiments, the layer comprising an additive comprises or is on at least an outer surface of the body.

In some embodiments, the layer comprising a lubricious additive is on or comprises at least 50, 55, 60, 65, 70, 75, 80, 85, 90, 95, 96, 97, 98 or at least 99% of the or each surface area of the body, preferably at least 75% or at least 90% of the or each surface area or between 75% and 100% of the or each surface area. In embodiments in which the layer comprising a lubricious additive comprises or is on both an inner and outer surface of the body, the additive may comprise at least 50, 55, 60, 65, 70, 75, 80, 85, 90, 95, 96, 97, 98 or at least 99% of each surface area of the body, preferably at least 75% or at least 90% of each surface area or between 75% and 100% of each surface area of both surfaces.

In some embodiments, at least 75% of the layer comprising a lubricious additive, or at least 76, 77, 78, 79, 80, 81, 82, 83, 84, 85, 86, 87, 88, 89, 90, 91, 92, 93, 94, 95, 96, 97, 98, 99, or 100% of the layer comprising a lubricious additive is the lubricious additive.

In some embodiments, the layer comprising a lubricious additive comprises an additive concentration of at least 0.1, 0.2, 0.3. 0.4. 0.5, 0.75, 1, 2, 3, 4, 5, 10, 15 or at least 20% by weight of the combination of base polymer and additive.

In some embodiments, the layer comprising a lubricious additive comprises an additive concentration of no greater than 70, 65, 60, 65, 60, 55, or of no greater than 50% by weight of the combination of the base polymer and additive.

The layer comprising a lubricious additive may comprise an additive concentration of greater than 5% by weight of the combination of base polymer and additive. The layer may comprise an additive concentration of between 6-50% by weight of the combination of base polymer and additive.

Using a high concentration of additive in the layer is beneficial, as it ensures that sufficient additive remains on the surface of the body even if some additive migrates out of the catheter. Furthermore, providing the additive as a separate layer from the body allows a high concentration to be used without significant undesired alteration/degradation of the base polymer mechanical properties.

However, providing the additive as a layer on or comprising the surface also allows for lower additive concentrations to be used overall, whilst still generating a lubricious surface, as the surface lubricity is not limited by the diffusion of additives to the surface from within the base polymer, which can often be non-ideal, and can be a problem of the prior art.

The layer comprising a lubricious additive may comprise an additive concentration of between 10-50% by weight of the combination of base polymer and additive, or of between 15-50, 20-50, 25-50, 30-50, 35-50, 40-50, or of between 45-50% by weight of the combination of base polymer and additive.

The layer comprising a lubricious additive may comprise an additive concentration of between 6-45% by weight of the combination of base polymer and additive, or of between 6-40, 6-35, 6-30, 6-25, 6-20, 6-15, or of between 6-10% by weight of the combination of base polymer and additive.

The layer comprising a lubricious additive may comprise an additive concentration of between 10-45% by weight of the combination of base polymer and additive, or of between 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, 15-20, or of between 10-15% by weight of the combination of base polymer and additive.

Having a layer comprising a lubricious additive on or comprising the surface of the body allows for high additive concentrations to be used without the additive substantially altering and/or degrading the mechanical properties of the intermittent catheter body.

In some embodiments, the layer comprising a lubricious additive comprises a thickness of at least 1 nm, or of at least 2, 3, 4, 5, 6, 7, 8, 9, 10, 15, 20, 25, 30, 35, 40, 45, or of at least 50 nm.

In some embodiments, the layer comprising a lubricious additive comprises a thickness of no more than 10000 nm, or of no more than 9000, 8000, 7000, 6000, 5000, 4000, 3000, 2000, 1000, 900, 800, 700, 600, 500, 400, or of no more than 300 nm.

In some embodiments, the layer comprising a lubricious additive comprises a thickness of between 50-300 nm.

The layer comprising a lubricious additive may comprise a thickness of between 60-300 nm, or of between 80-300, 100-300, 120-300, 140-300, 160-300, 180-300, 200-300, 220-300, 240-300, 260-300, or of between 280-300 nm.

The layer comprising a lubricious additive may comprise a thickness of between 50-280 nm, or of between 50-260, 50-240, 50-220, 50-200, 50-180, 50-160, 50-140, 50-120, 50-100, 50-80, or of between 50-60 nm.

The layer comprising a lubricious additive may comprise a thickness of between 60-280 nm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 μm.

In some embodiments, the body comprises a further lubricious additive. The further lubricious additive in the body may comprise the same lubricious additive as the layer comprising a lubricious additive. In other embodiments, the further lubricious additive in the body is different from the lubricious additive of the layer comprising a lubricious additive.

The further lubricious may comprise a concentration as described for the layer comprising an additive above.

The further lubricious additive may be concentrated at or on the surface of the body on which the layer comprising a lubricious additive is present. For example, at least 25%, 30%, 35%, 40%, 45%, 50%, 55%, 60%, 65%, 70%, 75% or at least 80% of the number of molecules of the further additive may be at or on the surface of the body.

In some embodiments, at least 25%, 30%, 35%, 40%, 45%, 50%, 55%, 60%, 65%, 70%, 75% or at least 80% of the number of molecules of further additive may have hydrophilic portions that are at or on the surface of the body.

In some embodiments, the further additive is located at and/or on at least 50, 55, 60, 65, 70, 75, 80, 85, 90, 95, 96, 97, 98 or at least 99% of the surface area of the body, preferably at least 75% or at least 90% of the surface area or between 75% and 100% of the surface area.

In other embodiments, the intermittent catheter comprises substantially no additive in the body, other than the part of the layer comprising a lubricious additive that may be melded or entangled with the base polymer, in such embodiments described above.

In some embodiments, one or both of the base polymer and the layer comprising a lubricious additive are independently cross-linked and/or the base polymer and the layer comprising a lubricious additive are cross-linked with each other.

The cross-links increase the difficulty for the additive to migrate from the layer and/or for any further additive in the body to migrate, particularly out of the intermittent catheter. This allows for the intermittent catheter to retain its lubricious surface for longer.

In some embodiments, the base polymer is cross-linked. In some embodiments, the base polymer is both independently cross-linked and cross-linked with the layer comprising an additive, and the layer comprising an additive is not independently cross-linked. In other embodiments, the base polymer is independently cross-linked but not cross-linked with the layer comprising an additive, and the layer comprising an additive is not independently cross-linked.

In some embodiments, the layer comprising an additive is cross-linked. In some embodiments, the layer comprising an additive is both independently cross-linked and cross-linked with the base polymer, and the base polymer is not independently cross- linked. In other embodiments, the layer comprising an additive is independently cross- linked but not cross-linked with the base polymer, and the base polymer is not independently cross-linked.

In some embodiments, the base polymer and layer comprising an additive are both independently cross-linked but not cross-linked with each other. In other embodiments, the base polymer and layer comprising an additive are not independently cross-linked but are cross-linked with each other. In other embodiments, both the base polymer and layer comprising an additive are independently cross-linked and cross-linked with each other.

In some embodiments, the base polymer is cross-linked at least at and/or on the outer surface. This is advantageous as it restricts migration of any further additive in the body out from the surface.

In some embodiments, the amphiphilic lubricious additive is polymeric or oligomeric.

In some embodiments, the additive is an A-B block copolymer comprising a hydrophobic hydrocarbon A-block and a hydrophilic B-block. The hydrophobic portion (A-block) may comprise a carbon chain of at least 5 carbon atoms, or at least 10, 15, 20, 25, 30, 35, or 40 carbon atoms. The hydrophobic portion may preferably comprise a carbon chain of between 20-52 carbon atoms.

In some embodiments, the A-block comprises a hydrocarbon chain block of the formula CH₃CH₂(CH₂CH₂)ₐ. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments, the B-block is a hydrophilic oligomer, i.e. a homo- or co-oligomer, comprising between 2 and 10 monomer units. The monomer units may be selected from the group comprising: alkylene oxides, alkylene glycols, epihalohydrins, unsaturated carboxylic acids, alkylene imines, lactones, vinyl alcohol, and vinyl alkanoates. The monomer units may be preferably selected from the group comprising: ethylene oxide, propylene oxide, ethylene glycol, propylene glycol, epichlorohydrin, acrylic acid, methacrylic acid, ethylene imine, caprolactone, vinyl alcohol, and vinyl acetate. In some embodiments the monomer units comprise alkylene oxide groups independently selected from ethylene oxide and propylene oxide, and in preferred embodiments all of the monomer units are ethylene oxide or all of the monomer units are propylene oxide.

The layer comprising a lubricious additive may comprise a single additive or may comprise a mixture of more than one additive.

In some embodiments, the additive (preferably the A-B block copolymer defined hereinabove) comprises poly(alkylene oxide) groups, preferably polyethylene oxide, and the additive is cross-linked through non-covalent bonds between the poly(alkylene oxide) groups and a complexing agent.

The complexing agent may be selected from one or more of the group comprising: a urea, a cyclodextrin, and a poly(unsaturated carboxylic acid), or combinations and/or derivatives thereof. The urea is preferably urea per se. The cyclodextrin may be selected from the group comprising: α-cyclodextrin, β-cyclodextrin, and γ-cyclodextrin; preferably α-cyclodextrin. The poly(unsaturated carboxylic acid) may preferably comprise poly(methacrylic acid) or a copolymer thereof. The copolymer may comprise a copolymer of poly(methacrylic acid) and an acrylate polymer, and preferably poly(methacrylic acid-co-methyl methacrylate).

In preferred embodiments, the intermittent catheter base polymer is hydrophobic or partly hydrophobic. A hydrophobic base polymer facilitates increased hydrophobic- hydrophobic interactions between the hydrophobic portion of the additive and the base polymer. This further decreases the energetic favourability for the hydrophobic portion to leave the base polymer and migrate out into the more hydrophilic external environment.

In some embodiments, the base polymer comprises a polymer selected from the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials.

In preferred embodiments, the base polymer comprises a polymer selected from the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials.

In some embodiments, said water disintegrable or enzymatically hydrolysable material comprises a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2- Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene- polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In other preferred embodiments, the base polymer comprises a polymer selected from the group comprising: polyolefins, polyvinyl chloride, polyurethane, styrene-butadiene copolymer (SBC), styrene-ethylene-butylene-styrene copolymer (SEBS), and thermoplastic elastomeric material or combinations, blends or copolymers of any of the above materials.

In some preferred embodiments, the base polymer comprises a polyolefin, especially polyethylene and/or polypropylene.

In some preferred embodiments, the base polymer comprises a thermoplastic elastomeric material. The base polymer may comprise a thermoplastic polyolefin.

The thermoplastic base polymer may comprise a hydrophobic polymer selected from the group comprising: Accurel™, Styroflex™, Styrolux™, MelifleX™, and Mediprene™.

The thermoplastic base polymer may comprise Estane™ 58315, which is both hydrophobic and hydrophilic.

In some embodiments, the base polymer comprises silane groups and the base polymer is cross-linked through Si—O—Si bonds between the silane groups and water. The silane groups may comprise trialkoxysilane groups or trialkylsilane groups.

The layer comprising a lubricious additive may comprise a separate or further lubricating agent or bacteria-repellent agent in addition to the additive. the separate of further lubricating agent or bacteria-repellent agent may be bonded to the layer.

In some embodiments, said further lubricating agent or bacteria-repellent agent comprises a material selected from the group comprising: silver-based, polytetrafluoroethylene, hydrogel, silicone, lecithin, salicylic acid, minocycline, rifampin, fluorinated ethylene propylene, polyvinylidone, polyvinyl compounds, polylactames, polyvinyl pyrrolidones, polysaccharides, heparin, dextran, xanthan gum, derivatised polysaccharides, hydroxy propyl cellulose, methyl cellulose, polyurethanes, polyacrylates, polyhydroxyacrylates, polymethacrylates, polyacrylamides, polyalkylene oxides, polyethylene oxides, polyvinyl alcohols, polyamides, polyacrylic acid, hydroxy ethylmethyl acrylate, polymethylvinyl ether, maleinic acid anyhydride, penicillin, neomycin sulfate, cephalothin, Bacitracin, phenoxymethyl penicillin, lincoymycin hydrochloride, sulfadiazine, methyl sulfadiazine, succinoylsulfathiazole, phthalylsulfathiazde, sulfacetamine, procaine penicillin, streptomycin, aureomycin, terramycin, terramycin, quaternary ammonium halides, cetyl pyridinium chloride, triethyl dodecyl ammonium bromide, hexachlorophene and nitrofurazone, or any combination thereof.

According to a second aspect of the invention, there is provided a method of manufacturing an intermittent catheter, the method comprising the step of extruding a base polymer and a lubricious additive to form a hollow polymeric tubular catheter body comprising the base polymer, and a layer comprising a lubricious additive on or comprising a surface of the catheter body.

The lubricious additive may comprise a hydrophilic or amphiphilic molecule. The additive may comprise any additive of the first aspect of the invention. The intermittent catheter may comprise any intermittent catheter of the first aspect of the invention.

Statements of invention relating to the intermittent catheter of the first aspect of the invention or to any of its components may also be applied to the second aspect of the invention.

In some embodiments, the method comprises co-extruding the base polymer and the layer comprising a lubricious additive simultaneously.

In some embodiments, the method comprises co-extruding the layer comprising an additive using direct, indirect, hydrostatic, or impact co-extrusion.

In some embodiments, the method comprises co-extruding the layer comprising an additive using cold, warm, hot, or friction co-extrusion.

In other embodiments, the method comprises extrusion coating the lubricious additive on the surface of the catheter body. The surface preferably comprises an outer surface of the body.

The base polymer and/or lubricious additive, preferably both, may be provided in granulate or powder form prior to extrusion.

The method may comprise melt-extruding the base polymer to form the hollow polymeric tubular body, and separately melt-extruding the lubricious additive onto a surface (preferably the outer surface) of the hollow polymeric tubular body.

The method may comprise using a blown or cast film process to coat the layer comprising an additive as a molten web of synthetic resin onto the surface of the intermittent catheter body after its formation.

The method may comprise extruding a molten layer comprising an additive from a slot die directly onto a tacky catheter body. The tacky catheter body may be moved beneath the die on extrusion coating of the additive to form the layer comprising an additive on the outer surface of the catheter.

Alternatively, the method may comprise co-extruding (melt extruding) both the base polymer and lubricious additive substantially simultaneously, so that a layer comprising a lubricious additive is formed as a layer on the base polymer, or comprises the surface of the base polymer.

The method may comprise melting both a base polymer mixture and lubricious additive and delivering a steady volumetric throughput of both the mixture and additive to a single extrusion head under pressure, which allows for co-extrusion of the layer comprising a lubricious additive and the base polymer simultaneously. The co-extrusion may employ elevated temperature and pressure causing entanglements to form between the base polymer chains and additive molecules.

The method may comprise mixing the granulate or powder base polymer with a further additive, as described in the first aspect of the invention, to form a mixture, and melt-extruding the mixture to form the hollow polymeric tubular body.

In some embodiments, the method comprises melting the mixture of the base polymer and further additive to form a second mixture before extruding the second mixture to form the hollow polymeric tubular intermittent catheter body.

In some embodiments, the method comprises the step of independently cross-linking one or both of the base polymer and the layer comprising a lubricious additive and/or cross-linking the base polymer and the layer comprising a lubricious additive with each other.

In some embodiments, the method comprises cross-linking the base polymer. In some embodiments, the method comprises both independently cross-linking the base polymer and cross-linking the base polymer with the layer comprising an additive. In other embodiments, method comprises independently cross-linking the base polymer but not cross-linking the base polymer with the layer comprising an additive.

In some embodiments, the method comprises independently cross-linking the layer comprising an additive. In some embodiments, the method comprises both independently cross-linking the layer comprising an additive and cross-linking the layer comprising an additive with the base polymer. In other embodiments, the method comprises independently cross-linking the layer comprising an additive but not cross-linking the layer comprising an additive with the base polymer.

In some embodiments, the method comprises independently cross-linking both the base polymer and the layer comprising an additive but not cross-linking the base polymer and layer comprising an additive with each other. In other embodiments, the method comprises cross-linking the base polymer and layer comprising an additive with each other but not independently cross-linking the base polymer or layer comprising an additive. In other embodiments, the method comprises independently cross-linking both the base polymer and layer comprising an additive and cross-linking the base polymer and the layer comprising an additive with each other.

In some embodiments, the method comprises cross-linking one or both of the base polymer and the layer comprising an additive independently before cross-linking the base polymer and the layer comprising an additive with each other.

In other embodiments, the method comprises cross-linking one or both of the base polymer and the layer comprising an additive independently after cross-linking the base polymer and the layer comprising an additive with each other.

In some embodiments, the method comprises cross-linking one or both of the base polymer and the layer comprising an additive independently at the same time as cross-linking the base polymer and the layer comprising an additive with each other.

In some embodiments the method comprises cross-linking only the base polymer or only the layer comprising an additive.

In some embodiments, the method comprises the step of cross-linking one or both of the base polymer and the layer comprising an additive independently and/or cross-linking the base polymer and the layer comprising an additive with each other during and/or after extrusion. Cross-linking may be performed during, after, or both during and after extrusion. The method may comprise cross-linking one or both of the base polymer and the layer comprising an additive independently during extrusion and cross-linking the base polymer and the layer comprising an additive with each other after extrusion.

Alternatively, the method may comprise cross-linking the base polymer and the layer comprising an additive with each other during extrusion and cross-linking one or both of the base polymer and the layer comprising an additive independently after extrusion.

In some embodiments, the method comprises the step of cross-linking one or both of the base polymer and the layer comprising an additive independently and/or cross-linking the base polymer and the layer comprising an additive with each other during and/or after extrusion of the layer comprising the additive on the surface of the base polymer.

In some embodiments, the method comprises the step of adding at least one cross-linker to one or both of the base polymer and the layer comprising an additive.

The at least one cross-linker may comprise one or both of an unsaturated monomer and a free radical.

The free radical may be produced by controlled/living radical polymerisation techniques. These techniques are known to a skilled person in the art and employ the principle of an equilibrium between free radicals and various types of dormant species (depending on the specific type of polymerisation technique employed.

The controlled/living radical polymerisation techniques include nitroxide-mediated polymerisation, reversible addition fragmentation transfer polymerisation (RAFT) and atom transfer radical polymerisation (ATRP). Anionic polymerisation may also be possible, although polymerisation is more sensitive to moisture and oxygen as impurities so may be less favourable than other controlled/living radical polymerisation processes.

More detailed descriptions of polymerisation mechanisms and related chemistry is discussed for nitroxide-mediated polymerisation (Chapter 10, pages 463 to 522), ATRP (Chapter 11, pages 523 to 628) and RAFT (Chapter 12, pages 629 to 690) in the Handbook of Radical Polymerization, edited by Krzysztof Matyjaszewski and Thomas P. Davis, 2002, published by John Wiley and Sons Inc.

When the polymer is prepared from anionic polymerisation techniques, initiators include, for example, hydrocarbyllithium initiators such as alkyllithium compounds (e.g., methyl lithium, n-butyl lithium, sec-butyl lithium), cycloalkyllithium compounds (e.g., cyclohexyl lithium and aryl lithium compounds (e.g., phenyl lithium, 1-methylstyryl lithium, p-tolyl lithium, naphyl lithium and 1,1-diphenyl-3- methylpentyl lithium. Also, useful initiators include naphthalene sodium, 1,4-disodio-1,1,4,4-tetraphenylbutane, diphenylmethyl potassium or diphenylmethylsodium.

The polymerisation process may also be carried out in the absence of moisture and oxygen and in the presence of at least one inert solvent. In one embodiment anionic polymerisation is conducted in the absence of any impurity which is detrimental to an anionic catalyst system. The inert solvent includes a hydrocarbon, an aromatic solvent or ether. Suitable solvents include isobutane, pentane, cyclohexane, benzene, toluene, xylene, tetrahydrofuran, diglyme, tetraglyme, orthoterphenyl, biphenyl, decalin or tetralin.

The anionic polymerisation process may be carried out at a temperature of 0° C. to −78° C.

A more detailed description of process to prepare polymers from an anionic process is discussed in Textbook of Polymer Science, edited by Fred W. Billmeyer Jr., Third Edition, 1984, Chapter 4, pages 88-90.

The controlled/living polymerisation processes leave a residue of reagent on the polymer chain such as (nitroxyl group from nitroxide-mediated), or a halogen from ATRP, thiocarbonylthio group from RAFT.

In one embodiment it is desirable to remove the residue i.e., remove the nitroxyl halogen or thiocarbonylthio group. Processes are known to a skilled person to remove such groups, and the disclosure in EP2791184 provides a solution to remove thiocarbonylthio groups. Other such techniques are described, for example, in Chong et at, Macromolecules 2007, 40, 4446-4455; Chong et al, Aust. J. Chem. 2006, 59, 755-762; Postma et al, Macromolecules 2005, 38, 5371-5374; Moad et al, Polymer International 60, no. 1, 2011, 9-25; and Wilcock et al, Polym. Chem., 2010, 1, 149-157.

In one embodiment The at least one cross-linker may comprise one or both of an unsaturated monomer and a nitroxyl free radical (common when nitroxide-mediated polymerisation is employed). The unsaturated monomer may comprise a monofunctional monomer and/or multifunctional monomer, such as a multifunctional olefin. The unsaturated monomer may be selected from the group comprising: acetylene, triallylisocyanurate (TAIC), trimethylol-propane-trimethacrylate (TMPTMA), triallylcyanurate (TAC), trimethylol-propane-triacrylate (TMPTA), hexakisalylaminocyclotriphosphazatrine (HAAP), maleic anhydride (MA), and poly(ethylene glycol) diacrylate (PEGDA), or combinations and/or derivatives thereof The nitroxyl free radical may comprise 2,2,6,6-tetramethylpiperidin-1-yl)oxyl free radical

(TEMPO) and/or a derivative thereof, which may optionally be selected from the group comprising: 4-hydroxy-2,2,6,6-tetramethylpiperidine-l-oxyl free radical (HO-TEMPO) and 4-benzoyloxy-2,2,6,6-tetramethylpiperidine-1 -oxyl free radical (BzO-TEMPO), or combinations and/or derivatives thereof

In ATRP polymerisation, groups that may be transferred by a radical mechanism include halogens (from a halogen-containing compound) or various ligands. A more detailed review of groups that may be transferred is described in U.S. Pat. No. 6,391,996.

Examples of a halogen-containing compound that may be used in ATRP polymerisation include benzyl halides such as p-chloromethylstyrene, α-dichloroxylene, α,α-dichloroxylene, α,α-dibromoxylene, hexakis(α-bromomethyl)benzene, benzyl chloride, benzyl bromide, 1-bromo-1-phenyl ethane and 1-chloro-1-phenylethane; carboxylic acid derivatives which are halogenated at the α-position, such as propyl 2-bromopropionate, methyl 2-chloropropionate, ethyl 2-chloropropionate, methyl 2-bromopropionate, and ethyl 2-bromoisobutyrate; tosyl halides such as p-toluenesulfonyl chloride; alkyl halides such as tetrachloromethane, tribromomethane, 1-vinylethyl chloride, and 1-vinylethyl bromide; and halogen derivatives of phosphoric acid esters, such as dimethylphosphoric acid.

In one embodiment when the halogen compound is employed, a transition metal such as copper is also present. The transition metal may be in the form of a salt. The transition metal is capable of forming a metal-to-ligand bond and the ratio of ligand to metal depends on the dentate number of the ligand and the co-ordination number of the metal. The ligand may be a nitrogen or phosphorus-containing ligand.

Examples of a suitable ligand include triphenylphosphine, 2,2-bipyridine, alkyl-2,2-bipyridine, such as 4,4-di-(5-heptyl)-2,2-bipyridine, tris(2-aminoethyl)amine (TREN), N,N,N′,N′,N″-pentamethyldiethylenetriamine, 4,4-do-(5-nonyl)-2,2-bipyridine, 1,1,4,7,10,10-hexamethyltriethylenetetramine and/or tetramethylethylenediamine. Further suitable ligands are described in, for example, International Patent application WO 97/47661. The ligands may be used individually or as a mixture. In one embodiment the nitrogen containing ligand is employed in the presence of copper. In one embodiment the ligand is phosphorus-containing with triphenyl phosphine (PPh₃) a common ligand. A suitable transition metal for a triphenyl phosphine ligand includes Rh, Ru, Fe, Re, Ni or Pd.

In RAFT polymerisation, chain transfer agents are important. A more detailed review of suitable chain transfer agents RAFT polymerisation, as described in International Patent Publication Nos. WO 98/01478, WO 99/31144 and WO 10/83569, is a polymerisation technique that exhibits characteristics associated with living polymerisation.

Examples of a suitable RAFT chain transfer agent include benzyl 1-(2-pyrrolidinone)carbodithioate, benzyl(1,2-benzenedicarboximido) carbodithioate, 2-cyanoprop-2-yl 1-pyrrolecarbodithioate, 2-cyanobut-2-yl l-pyrrolecarbodithioate, benzyl 1-imidazolecarbodithioate, N,N-dimethyl-S-(2-cyanoprop-2-yl)dithiocarbamate, N,N-diethyl-S-benzyl dithiocarbamate, cyanomethyl 1-(2-pyrrolidone) carbodithoate, cumyl dithiobenzoate, 2-dodecylsulphanylthiocarbonylsulphanyl-2-methyl-propionic acid butyl ester, O-phenyl-S-benzyl xanthate, N,N-diethyl S-(2-ethoxy-carbonylprop-2-yl)dithiocarbamate, dithiobenzoic acid, 4-chlorodithiobenzoic acid, O-ethyl-S-(1-phenylethyl)xanthtate, O-ethyl-S-(2-(ethoxycarbonyl)prop-2-yl)xanthate, O-ethyl-S-(2-cyanoprop-2-yl)xanthate, O-ethyl-S-(2-cyanoprop-2-yl)xanthate, O-ethyl-S-cyanomethyl xanthate, O-pentafluorophenyl-S-benzyl xanthate, 3-benzylthio-5,5-dimethylcyclohex-2-ene-1-thione or benzyl 3,3-di(benzylthio)prop-2-enedithioate, S,S′-bis-(α,α′-disubstituted-α″-acetic acid)-trithiocarbonate, S,S′-bis-(α,α′-disubstituted-α″-acetic acid)-trithiocarbonate or S-alkyl-S′-(α,α′-disubstituted-α″-acetic acid)-trithiocarbonates, benzyl dithiobenzoate, 1-phenylethyl dithiobenzoate, 2-phenylprop-2-yl dithiobenzoate, 1-acetoxyethyldithiobenzoate, hexakis(thiobenzoylthiomethyl)benzene, 4-bis(thiobenzoylthiomethyObenzene, 1,2,4,5-tetrakis(thiobenzoylthiomethyl)benzene, 1,4-bis-(2-(thiobenzoylthio)-prop-2-yl)benzene, 1-(4-methoxyphenyl)ethyl dithiobenzoate, benzyl dithioacetate, ethoxycarbonylmethyl dithioacetate, 2-(ethoxycarbonyl)prop-2-yl dithiobenzoate, 2,4,4-trimethylpent-2-yl dithiobenzoate, 2-(4-chlorophenyl)prop-2-yl dithiobenzoate, 3-vinylbenzyl dithiobenzoate, 4-vinylbenzyl dithiobenzoate, S-benzyl diethoxyphosphinyldithioformate, tert-butyl trithioperbenzoate, 2-phenylprop-2-yl 4-chlorodithiobenzoate, 2-phenylprop-2-yl 1-dithionaphthalate, 4-cyanopentanoic acid dithiobenzoate, dibenzyl tetrathioterephthalate, dibenzyl trithiocarbonate, carboxymethyl dithiobenzoate or poly(ethylene oxide) with dithiobenzoate end group or mixtures thereof.

In one embodiment a suitable RAFT chain transfer agent includes 2-Dodecylsulfanylthiocarbonylsulfanyl-2-methyl-propionic acid butyl ester, cumyl dithiobenzoate or mixtures thereof.

A discussion of the polymer mechanism of RAFT polymerisation is shown on page 664 to 665 in section 12.4.4 of Matyjaszewski et al.

The method may comprise adding the at least one cross-linker to one or both of the base polymer and the layer comprising an additive before or during extrusion of the intermittent catheter.

In some embodiments, the method comprises the step of adding at least one cross-linking activator to one or both of the base polymer and the layer comprising an additive.

The method may comprise adding both at least one cross-linking activator and at least one cross-linker to one or both of the base polymer and the layer comprising an additive.

The at least one cross-linking activator may activate cross-linking of the base polymer, the layer comprising an additive, and/or of the base polymer with the layer comprising an additive.

The at least one cross-linking activator may comprise a radical initiator. The radical initiator may comprise a thermal radical initiator and/or a photo-radical initiator. The radical initiator may comprise a peroxide. The peroxide may be selected from the group comprising: benzoyl peroxide (BPO), di-tent-butyl peroxide, cumene hydroperoxide, tert-butyl hydroperoxide, 2,5-bis(tert-butylperoxy)-2,5-dimethylhexane (DHBP), di(tert-butylperoxyisopropyObenzene, dicumyl peroxide (DCP), 2,5-di(tert-butylperoxy)-2,5-dimethyl-3-hexyne (DTBPHY) or combinations and/or derivatives thereof. The radical initiator may comprise an azo compound. The azo compound may be selected from the group comprising: AIBN, AMBN, ADVN, ACVA, dimethyl 2,2′-azobis(2-methylpropionate), AAPH, and 2,2′-azobis[2-(2-imidazolin-2-yl)-propane] dihydrochloride, or combinations and/or derivatives thereof The photo-radical initiator may be selected from the group comprising: camphorquinone, acetophenone, 3-acetophenol, 4-acetophenol, benzophenone, 2-methylbenzophenone, 3-methylbenzophenone, 3-hydroxybenzophenone, 3,4-dimethyl benzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis(diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone (Irgacure 2959), 1-benzoylcyclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio) morpholinopropiophenone, 2-benzyl-2-(dimethylamino)-4′-morpholinobutyrophenone, 2-isonitrosopropiophenone, anthraquinone, 2-ethylanthraquinone, sodium anthraquinone-2-sulfonate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′-bis(2-chlorophenyl)-4,4′,5,5′-tetraphenyl-1,2′-biimidazole, diphenyl(2,4,6-trimethylbenzoyl)phosphine oxide, phenylbis(2,4,6-trimethylbenzoyl)phosphine oxide, and lithium phenyl(2,4,6-trimethylbenzoyl)phosphinate, or combinations and/or derivatives thereof.

The free radical initiator may be present at 0.01 to 5 wt %, 0.5 to 2 wt % based on the total weight of the monomers used to prepare the crosslinked polymer disclosed herein,

The molar ratio of free radical initiator to the reagent living/controlled polymerisation radical agent may range from 0.05 to 1:1, or 0.2:1 to 0.8:1, or 0.3 to 0.5:1.

The method may be carried out as a batch process, a semi-batch process, a continuous process, a feed process or a bulk process. The process may be in an emulsion, in solution or suspension.

The method may comprise adding the at least one cross-linking activator to one or both of the base polymer and the layer comprising an additive before or during extrusion of the intermittent catheter.

The method may comprise adding the at least one cross-linker to one or both of the base polymer and the layer comprising an additive before extrusion of the intermittent catheter and adding the at least one cross-linking activator to one or both of the base polymer and the layer comprising an additive after extrusion. This may comprise soaking the extruded intermittent catheter in a peroxide-containing solution, allowing the peroxide to diffuse into the catheter.

The method may comprise cross-linking the base polymer and/or layer comprising an additive by packaging the intermittent catheter in or with a cross-linking inducing medium.

Having the additive present as a layer, especially on or comprising the outer surface of the catheter body, has the further advantage of limiting the depth of penetration required by the cross-linking inducing medium to initiate cross-linking of the layer comprising an additive.

The method may comprise packaging the intermittent catheter in a container comprising a solution comprising the at least one cross-linking activator to induce cross-linking.

In some embodiments, the method comprises adding both MA cross-linker and DHBP cross-linking activator to one or both of the base polymer and the layer comprising an additive.

In some embodiments, the method comprises adding both HO-TEMPO cross-linker and di(tert-butylperoxyisopropyl)benzene cross-linking activator to one or both of the base polymer and the layer comprising an additive.

In some embodiments, the method comprises adding both BzO-TEMPO cross-linker and di(tert-butylperoxyisopropyl)benzene cross-linking activator to one or both of the base polymer and the layer comprising an additive.

In some embodiments, the method comprises adding both TMPTA cross-linker and DHBP cross-linking activator to one or both of the base polymer and the layer comprising an additive.

In some embodiments, the method comprises adding at least one cross-linking co-monomer to one or both of the base polymer and the layer comprising an additive.

The method may comprise adding both at least one cross-linking co-monomer and at least one cross-linker; both at least one cross-linking co-monomer and at least one cross-linking activator; or the method may comprise adding at least one cross-linking co-monomer, at least one cross-linker, and at least one cross-linking activator to one or both of the base polymer and the layer comprising an additive.

The at least one cross-linking co-monomer may be selected from the group comprising: an N-halosuccinimide (such as N-bromosuccinimide), a furan derivative, and butyl 3-(2-thienyl) propenoate (BTA), or combinations and/or derivatives thereof. The furan derivative may comprise a furan nitrile derivative, which may comprise 2-(furan-2-ylmethylene)malononitrile (FN).

The method may comprise adding the at least one cross-linking co-monomer to one or both of the base polymer and the layer comprising an additive before extrusion of the intermittent catheter. Adding at least one cross-linking co-monomer to one or both of the base polymer and the layer comprising an additive may assist in limiting chain scission of the base polymer and/or the additive leading to improved catheter material properties.

In some embodiments, the method comprises the step of cross-linking by irradiating one or both of the base polymer and the layer comprising an additive.

Having the additive present as a layer, especially on or comprising the outer surface of the catheter body, has the further advantage of limiting the depth of penetration required by radiation to initiate cross-linking of the layer comprising an additive.

The method may comprise irradiating with E-beam and/or γ radiation.

The method may comprise irradiating one or both of the base polymer and the layer comprising an additive after addition of one or more of the groups comprising: the at least one cross-linker, the at least one cross-linking activator, the at least one cross-linking co-monomer, and combinations thereof.

The method may comprise irradiating the intermittent catheter after extrusion to cross-link the catheter surface. Irradiating one or both of the base polymer and the layer comprising an additive may sterilise the intermittent catheter as well as encourage cross-linking. Irradiating for sterilisation and cross-linking at the same time may be advantageous in reducing the number of operations required in manufacturing the intermittent catheter.

The method may comprise adding at least one inorganic additive or filler to one or both of the base polymer and the layer comprising an additive before irradiation cross-linking. Inorganic additives and fillers are believed to provide the advantage of increasing the number of radicals generated during irradiation cross-linking. The inorganic additive or filler may comprise one or more of the group comprising: TiO₂, CaCO₃, talc, and clay, or combinations thereof.

In some embodiments, the method comprises the steps of grafting silane groups to the base polymer; and adding water to the base polymer to cross-link the silane groups.

The base polymer may preferably comprise a polyolefin.

The step of grafting silane groups to the base polymer may comprise reacting the base polymer with an organofunctional silane.

The organofunctional silane may comprise one or more functional groups of the list comprising: an unsaturated group, a thiol, an amine, and an epoxide. The organofunctional silane may be selected from the group comprising: vinyltrimethoxysilane (VMSI), vinyltriethoxysilane (VESI), 3-methacryloyloxypropyl-trimethoxysilane (MMSI), 3-mercaptopropyl-trimethoxysilane (SMSI), 3-aminopropyl-trimethoxysilane (NMSI), and 3-glycidyloxypropyl-trimethoxysilane (GMSI), or combinations and/or derivatives thereof.

The method may comprise adding the organofunctional silane in a concentration of at least 0.1, 0.2, 0.3, 0.4, 0.5, 0.75, 1, 2, 3, 4, 5, 6, 7, 8, 9, or at least 10% by weight of the base polymer. The method may comprise adding the organofunctional silane in a concentration of no more than 40, 35, 30, 25, 20, or 15% by weight of the base polymer. A preferred concentration may be between 0.1% and 10% by weight of the base polymer, and more preferably between 1% and 5%, or at a concentration of 2% by weight of the base polymer.

The reaction of the base polymer with the organofunctional silane may be performed in the presence of the at least one cross-linking activator. The at least one cross-linking activator may comprise a radical initiator as listed above.

The method may comprise adding the at least one cross-linking activator in a concentration of at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or at least 2% by weight of the base polymer. The method may comprise adding the at least one cross-linking activator in a concentration of no more than 10% by weight of the base polymer, or no more than 9, 8, 7, 6, 5, 4, or 3% by weight of the base polymer. A preferred concentration may be between 0.01% and 2% by weight of the base polymer, and more preferably between 0.05% and 1%, or at a concentration of around 0.1% to 0.5% by weight of the base polymer.

The step of grafting silane groups to the base polymer may be performed during extrusion of the base polymer.

The step of grafting silane groups to the base polymer may be performed at an elevated temperature of at least 50° C., or at least 60, 70, 80, 90, 100, 110, 120, 130, 140, 150, 160, 170, 180, 190, 200, 210, 220, 230, 240, 250, 260, 270, 280, 290, or at least 300° C. This step may be performed at a temperature of between 100° C. and 300° C., and preferably between 140° C. and 240° C.

The step of cross-linking the silane groups may be performed by adding water after extrusion of the base polymer.

The step of adding water to the base polymer to cross-link the silane groups may be performed at a temperature of at least 20° C., or at least 30, 40, 50, 60, 70, 80, 90, 100, 110, 120, 130, 140, or at least 150° C. This step may be performed at a temperature of between 50° C. and 150° C., preferably between 60° C. and 90° C.

The step of adding water to the base polymer may be performed in a steam chamber of a hot water tank.

The step of cross-linking the silane groups may comprise adding a catalyst, preferably before adding water to the base polymer. The catalyst may be added during extrusion of the base polymer. The catalyst may comprise an organotin derivative, such as dibutyltindilaurate.

The silane groups may be cross-linked for at least 5, 10, 15, 20, 25, or at least 30 minutes, or for at least 1, 2, 3, 4, 5, 6, 7, 8, 9, 10, 12, 14, 16, 18, 20, or at least 22 hours, or for at least 1 day, or at least 1 day and 5, 10, 15, 20 hours, or for at least 2 days.

In some embodiments, the base polymer is functionalised with reactive side chains and the method comprises the step of cross-linking the base polymer independently and/or with the layer comprising an additive by reacting the reactive side chains with each other and/or with functional groups on the additive.

Cross-linking may be performed by irradiating the functionalised base polymer, preferably with ultraviolet radiation.

Cross-linking may be performed at a temperature of at least 10° C., or at least 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or at least 400° C. Cross-linking may be performed at a temperature of between 20° C. and 300° C., preferably between 30° C. and 200° C.

Cross-linking may be performed for at least 5, 10, 15, 20, 25, or at least 30 minutes, or for at least 1, 2, 3, 4, 5, 6, 7, 8, 9, 10, 12, 14, 16, 18, 20, or at least 22 hours, or for at least 1 day, or at least 1 day and 5, 10, 15, 20 hours, or for at least 2 days.

The functionalised base polymer may preferably comprise a polyolefin functionalised with reactive side chains. The reactive side chains may be selected from the group comprising: an unsaturated group, an epoxide, a borane, and combinations thereof. The functionalised base polymer may comprise a polyolefin co- or terpolymer comprising the reactive side chains. The reactive side chains may be incorporated onto the polyolefin chain through a metallocene catalysed reaction using reactive co-monomers comprising the reactive side chains. The reactive co-monomer may be selected from the group comprising: a vinylbenzene derived olefin, a 9-BBN derived olefin, a methylbenzene derived olefin, a glycidyl derived olefin, and combinations thereof. The functionalised base polymer may be selected from the group comprising: poly(ethylene-ter-propylene-ter-divinylbenzene) (EP-DVB), poly(ethylene-ter-1-octene-ter-divinylbenzene) (EO-DVB), poly(ethylene-co-glycidyl methacrylate), and combinations thereof.

In some embodiments, the layer comprising an additive comprises poly(alkylene oxide) groups and the method comprises the step of forming non-covalent bonds between the poly(alkylene oxide) groups and a complexing agent to cross-link the layer comprising an additive.

The complexing agent may be selected from the group comprising: a urea, a cyclodextrin, and a poly(unsaturated carboxylic acid), or combinations and/or derivatives thereof. The urea is preferably urea per se. The cyclodextrin may be selected from the group comprising: α-cyclodextrin, β-cyclodextrin, and γ-cyclodextrin; preferably α-cyclodextrin. The poly(unsaturated carboxylic acid) may preferably comprise poly(methacrylic acid) or a copolymer thereof. The copolymer may comprise a copolymer of poly(methacrylic acid) and an acrylate polymer, and preferably poly(methacrylic acid-co-methyl methacrylate).

The method may comprise packaging the intermittent catheter in a container comprising a solution of the complexing agent to form the non-covalent bonds between the poly(alkylene oxide) groups and the complexing agent.

According to a third aspect of the invention, there is provided the use of a layer comprising a lubricious additive comprising an amphiphilic molecule on a surface of an intermittent catheter as a lubricating agent.

The lubricious additive may comprise any additive of the first aspect of the invention.

The intermittent catheter may comprise any intermittent catheter of the first aspect of the invention. Statements of invention relating to the intermittent catheter of the first aspect of the invention or to any of its components may also be applied to the third aspect of the invention.

According to a fourth aspect of the invention, there is provided a packaged intermittent catheter of the first aspect of the invention comprising a packaging container in which is located an intermittent catheter of the first aspect of the invention, and optionally a wetting agent.

The wetting agent may surround the intermittent catheter or may be separated from the intermittent catheter within the packaging, for example by providing the wetting agent in a separate container within the packaging container.

### DETAILED DESCRIPTION OF THE INVENTION

In order that the invention may be more clearly understood embodiments thereof will now be described, by way of example only:

### Example 1

A first embodiment of an intermittent catheter of the invention comprises an intermittent catheter comprising a hollow polymeric tubular body comprising a base polymer formed of polyethylene and further comprising an amphiphilic additive of the formula CH₃CH₂(CH₂CH₂)₂₀(OCH₂CH₂)₈OH present as a layer comprising the complete outer surface of the body of the catheter. The layer comprising an additive is physically entangled and melded with the body, holding it in place. The amphiphilic additive comprises a hydrophilic block which seeks away from the body and towards the external environment due to its incompatibility with the base polymer, the outer surface becoming lubricious as a result. The lipophilic and hydrophobic block of the amphiphilic additive further ensures that the hydrophilic block is secured to the base material.

The intermittent catheter may be prepared as described in US patents U.S. Pat. Nos. 10,058,638 B2 and 9,186,438 B2, but without adding the additive to the base polymer mixture and with an added co-melt extrusion step. This step involves melting both the base polymer mixture and lubricious additive and delivering a steady volumetric throughput of both the mixture and additive to a single extrusion head under pressure, which allows for co-extrusion of the layer comprising a lubricious additive and the base polymer simultaneously. The high temperature and pressure employed in the co-extrusion step allows for entanglements to form between the base polymer polyethylene chains and the additive molecules. After co-extrusion, the catheter is cooled to allow the catheter body to solidify.

The layer comprising an additive comprises a thickness of 70 μm and an additive concentration of 3% by weight of the combination of the base polymer and the layer comprising an additive.

The intermittent catheter is used in the conventional manner.

The layer comprising an amphiphilic additive on the outer surface of the catheter body confers higher lubricity to the outer surface of the intermittent catheter than conventional intermittent catheters of the prior art, making it both easier to insert and remove, even with the additive being present in a low concentration. Despite the additive being entirely on the outer surface of the catheter, the melds and entanglements between the base polymer and the layer comprising an additive mean that migration of the additive away from the catheter surface is comparable to or even lower than conventional intermittent catheters of the prior art.

### Example 2

A second embodiment of an intermittent catheter of the invention comprises an intermittent catheter comprising a hollow polymeric tubular body comprising a base polymer formed of thermoplastic polypropylene and further comprising an amphiphilic additive of the formula CH₃CH₂(CH₂CH₂)₁₅(OCH₂CH₂)₅OH present in the body and as a layer comprising the lubricious additive on the complete outer surface of the body of the catheter. The amphiphilic additive comprises a hydrophilic block which seeks away from the body and towards the external environment due to its incompatibility with the base polymer, the outer surface becoming lubricious as a result. The lipophilic and hydrophobic block of the amphiphilic additive ensures that the hydrophilic block is secured to the base material.

The intermittent catheter may be prepared as described in US patents U.S. Pat. Nos. 10,058,638 B2 and 9,186,438 B2, but with the added step of extrusion coating the layer comprising a lubricious additive on the outer surface of the catheter body after forming the body. This step can be performed using a blown or cast film process to coat the layer comprising an additive as a molten web of synthetic resin onto the outer surface of the intermittent catheter body after its formation. The process involves extruding the molten layer comprising an additive from a slot die directly onto a tacky catheter body that is moved beneath the die to form the layer on the outer surface of the catheter. The catheter is then cooled to bring the molten film of additive back into a solid/gel state and to completely solidify the tacky catheter body.

The layer comprising an additive comprises a thickness of 100 μm and an additive concentration of 6% by weight of the combination of the base polymer and the layer comprising an additive.

The intermittent catheter is used in the conventional manner.

The layer comprising an amphiphilic additive confers high lubricity to the outer surface of the intermittent catheter and the further additive in the body also seeks towards the outer surface of the catheter and enhances the surface lubricity. Accordingly, the catheter comprises much higher lubricity on its outer surface than conventional intermittent catheters of the prior art, making it both easier to insert and remove.

### Example 3

A third embodiment of an intermittent catheter of the invention comprises an intermittent catheter comprising a hollow polymeric tubular body comprising a base polymer formed of thermoplastic polyethylene and further comprising an amphiphilic additive of the formula CH₃CH₂(CH₂CH₂)₁₀(OCH₂CH₂)₄OH present as a layer comprising the complete outer surface of the body of the catheter. The amphiphilic additive confers lubricity to the outer surface. The lipophilic and hydrophobic block of the amphiphilic additive helps secure it to the base material and the layer comprising an additive is also physically entangled with the body.

The layer comprising an additive is also independently cross-linked through non-covalent bonds formed between the poly(ethylene oxide) groups and a urea complexing agent.

The non-cross-linked intermittent catheter may be prepared as described in Example 1. The layer comprising an amphiphilic additive may be independently cross-linked by packaging the non-cross-linked intermittent catheter submerged in a urea cross-linking inducing solution.

Having the additive present as a layer comprising the outer surface of the catheter body has the advantage of limiting the depth of penetration required by the cross-linking inducing medium to initiate the cross-linking.

The layer comprising an additive comprises a thickness of 70 μm and an additive concentration of 3% by weight of the combination of the base polymer and the layer comprising an additive.

The intermittent catheter is used in the conventional manner.

The layer comprising an amphiphilic additive on the outer surface of the catheter body confers high lubricity to the outer surface of the intermittent catheter even with a low additive concentration, as in Example 1.

The additive cross-links increase the difficulty for the additive to migrate, particularly out of the intermittent catheter. Cross-linking is believed to reduce the mobility of the polymer matrix, which in turn restricts the migration of the additive out of the catheter. This allows the intermittent catheter to retain its lubricity for longer even when packaged in water or aqueous solutions.

The intermittent catheter of Example 3 conferred reduced migration of the amphiphilic additive from the surface of the catheter during both storage/transport and through use of the catheter. It also provided increased resistance to abrasion of the additive from the surface of the catheter on contact with external bodies.

### Example 4

A fourth embodiment of an intermittent catheter of the invention comprises the intermittent catheter of Example 1 packaged in a packaging container with a wetting agent.

The intermittent catheter is produced as detailed in Example 1. After formation of the coated catheter, it was packaged in a sterile sachet comprising a saline solution wetting agent. The intermittent catheter is fully submerged in the saline solution, which allows for activation of the lubricious outer surface of the catheter due to hydrophilic-hydrophilic interactions between the hydrophilic block of the amphiphilic additive and the hydrophilic wetting agent. The hydrophilic-hydrophilic interactions ensure that the hydrophilic blocks of the additive molecules seek away from the body and towards the external environment, generating a highly lubricious outer surface.

Due to the additive being entirely on the outer surface of the catheter, the wetting agent is able to generate lubricious outer surface instantaneously, and which will be active as soon as a health care professional or user removes the catheter.

The above embodiments are described by way of example only. Many variations are possible without departing from the scope of the invention as defined in the appended claims.

1. An intermittent catheter comprising a hollow polymeric tubular body comprising a base polymer and a layer comprising a lubricious additive on or comprising a surface of the body, wherein the lubricious additive comprises an amphiphilic molecule.

2. An intermittent catheter as claimed in claim 1, wherein the surface comprises an outer surface of the body.

3. An intermittent catheter as claimed in claim 2, wherein the layer comprising a lubricious additive is on or comprises at least 75% of the outer surface area of the body.

4. An intermittent catheter as claimed in claim 1, wherein at least 75% of the layer comprising a lubricious additive is the lubricious additive.

5. An intermittent catheter as claimed in claim 1, wherein the layer comprising a lubricious additive comprises an additive concentration of greater than 5% by weight of the combination of base polymer and layer comprising a lubricious additive.

6. An intermittent catheter as claimed in claim 1, wherein the layer comprising a lubricious additive comprises a thickness of between 50 and 300 μm.

7. An intermittent catheter as claimed in claim 1, wherein the body comprises a further lubricious additive.

8. An intermittent catheter as claimed in claim 7, wherein the further lubricious additive comprises the same lubricious additive as the layer comprising a lubricious additive.

9. An intermittent catheter as claimed in claim 1, wherein the layer comprising a lubricious additive is cross-linked.

10. An intermittent catheter as claimed in claim 1, wherein the amphiphilic additive is an amphiphilic A-B block copolymer comprising a hydrophobic hydrocarbon A-block and a hydrophilic B-block.

11. An intermittent catheter as claimed in claim 10, wherein the amphiphilic additive is an A-B block copolymer comprising an A-block comprising a hydrocarbon chain block of the formula CH₃CH₂(CH₂CH₂)ₐ where “a” is 5-25 and a hydrophilic B-block.

12. An intermittent catheter as claimed in claim 10, wherein the B-block is a hydrophilic oligomer comprising between 2 and 10 monomer units derived from one or more monomers selected from the group consisting of: alkylene oxides, alkylene glycols, epihalohydrins, unsaturated carboxylic acids, alkylene imines, lactones, vinyl alcohol, and vinyl alkanoates.

13. (canceled)

14. (canceled)

15. A method of manufacturing an intermittent catheter, the method comprising the step of extruding a base polymer and a lubricious additive to form a hollow polymeric tubular catheter body comprising the base polymer, and a layer comprising a lubricious additive on or comprising a surface of the catheter body.

16. A method as claimed in claim 15, wherein the method comprises co-extruding the base polymer and the layer comprising a lubricious additive simultaneously.

17. A method as claimed in claim 15, wherein the method comprises extrusion coating the lubricious additive on the surface of the catheter body.

18. A method as claimed in claim 15, wherein the method further comprises the step of cross-linking the layer comprising an additive.

19. A method as claimed in claim 15, wherein the layer comprising an additive comprises a hydrophilic or amphiphilic molecule.

20. A method as claimed in claim 19, wherein the amphiphilic additive is an amphiphilic A-B block copolymer comprising a hydrophobic hydrocarbon A-block and a hydrophilic B-block.

21. A method as claimed in claim 20, wherein the amphiphilic additive is an A-B block copolymer comprising an A-block comprising a hydrocarbon chain block of the formula CH₃CH₂(CH₂CH₂)ₐ where “a” is 5-25, and a hydrophilic B-block.

22. A method as claimed in claim 20, wherein the B-block is a hydrophilic oligomer comprising between 2 and 10 monomer units derived from one or more monomers selected from the group consisting of: alkylene oxides, alkylene glycols, epihalohydrins, unsaturated carboxylic acids, alkylene imines, lactones, vinyl alcohol, and vinyl alkanoates.

23-26. (Canceled)
